(19) United States
(12) Patent Application Publication
(10) Pub. No.: US 2004/0123349 A1
(43) Pub. Date: Jun. 24, 2004

(54) SINAT5, an Arabidopsis thaliana gene promotes ubiquitin related degradation

(76) Inventors: Qi Xie, Singapore (SG); Hui Shan Guo, Singapore (SG); Nam Hai Chua, New York, NY (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/324,120

(22) Filed: Dec. 20, 2002

(51) Int. Cl. A01H 1/00; C12N 15/82

(52) U.S. Cl. 800/287

(57) ABSTRACT

The present invention is directed to methods of growing a transgenic plant comprising transforming a plant with a nucleic acid encoding the SINAT5 polypeptide of Arabidopsis thaliana. SINAT5 is an E3 ubiquitin ligase which regulates NAC1 gene expression and plant growth. Mutations in the SINAT5 polypeptide are also provided.

### FIELD OF THE INVENTION

The present invention is related to genetic regulation of development in plants. More specifically, the invention is related to the Arabidopsis gene SINAT5 which regulates ubquitylation of certain plant proteins and development in plants. The invention is also related to transgenic plants expressing a mutant or recombinant SINAT5 gene.

The publications and other materials used herein to illuminate the background of the invention or provide additional details respecting the practice are incorporated by reference and for ease of reference are included in the Bibliography.

### BACKGROUND AND SIGNIFICANCE

IAA (or auxin) controls many important aspects of plant development including the initiation and elongation of lateral roots. Genetic screens have uncovered several Arabidopsis mutants that are blocked in auxin signaling and these mutants also produce fewer lateral roots¹⁻³. Molecular and biochemical characterization of the proteins affected in these mutants show that ubiquitin-mediated proteolysis plays a central role in this process. In particular, the AXR₁ and TIR1 genes are needed for the assembly of an active SCF complex that likely promotes the degradation of AUX/IAA transcriptional repressors in response to auxin⁴⁻⁸.

Recent work has highlighted the importance of regulated proteolysis in auxin signaling events^{6,7}. In the absence of auxin, the target genes for auxin signal transduction are presumably repressed by AUX/IAA transcriptional regulators. Upon exposure to the hormone, however, these repressors are degraded by ubiquitin-dependent proteolysis through the SCF complex of which TIR1 is an integral subunit, leading to an up-regulation of auxin signals.

Numerous biological processes are controlled by the ubiquitination of cellular protein in eukaryotic organisms. Cellular processes that are affected by ubiquitin modification include the regulation of gene expression, regulation of the cell cycle and cell division, cellular housekeeping, cell-specific metabolic pathways, disposal of mutated or post-translationally damaged proteins, the cellular stress response, modification of cell surface receptors, DNA repair, import of proteins into mitochondria, apoptosis, and growth factor-mediated signal transduction.

The ubiquitin proteolytic pathway requires the covalent attachment of ubiquitin (E1), a highly conserved 76 amino acid protein, to defined lysine residues of substrate proteins. Substrate recognition by this pathway involves a specialized recognition and targeting apparatus, the ubiquitin-conjugating system. Ubiquitin-conjugating enzyme (E2) and ubiquitin-protein ligase (E3), either independently or in conjunction, catalyze isopeptide formation between the carboxyl terminus of ubiquitin and amino groups of internal lysine residues of target proteins. Ubiquitin-protein conjugates are then recognized and degraded by a specific protease complex, the 26S proteasome. Both E2 and E3 exist as protein families, and their pattern of expression is thought to determine substrate specificity.

Several RING motif proteins have recently been shown to function as E3s in vitro⁹⁻¹¹. The Drosophila SINA protein has been shown to promote degradation of the cell-fate repressor TRAMTRACK¹². Mammalian SIAHs are known to control the stability of the membrane receptor, DCC (Deleted in Colorectal Cancer)¹³, several transcription factors and other proteins¹⁴⁻¹⁹.

It has previously been demonstrated that NAC1, a NAM/CUC family member, is a transcription activator that functions downstream of TIR1 to transduce the auxin signal for lateral root development²⁰. In transgenic plants, overexpression of NAC1 produced more lateral roots whereas under-expression of NAC1 produced fewer lateral roots.

### SUMMARY OF THE INVENTION

The invention provides an isolated nucleic acid encoding a polypeptide selected from the group consisting of SEQ ID NO:1, SEQ ID NO:2 and SEQ ID NO:3 and an isolated nucleic acid encoding the polypeptide of SEQ ID NO:1 as shown in SEQ ID NO:4. The invention also provides an isolated polypeptide comprising an amino acid sequence as set forth in SEQ ID NO:1, SEQ ID NO:2 or SEQ ID NO:3. The invention also provides a vector comprising a nucleic acid of the invention, a cell transformed with a nucleic acid of the invention that in one embodiment may be a plant cell, a plant transformed with a nucleic acid of the invention and a plant grown from a plant cell transformed with a nucleic acid of the invention.

In one embodiment, the invention provides a transgenic plant cell comprising a nucleic acid encoding the polypeptide of SEQ ID NO:1, wherein the polypeptide is overexpressed in said cell when compared to a wildtype plant cell comprising a nucleic acid encoding SEQ ID NO:1.

In another embodiment, the invention provides a method of growing a transgenic plant which comprises transforming a plant or plant cell with a nucleic acid of the invention encoding the wildtype SINAT5 polypeptide of SEQ ID NO:1, wherein the nucleic acid and wildtype SINAT5 polypeptide is expressed during the growth of the plant at a level greater than the level of expression of any endogenous wildtype SINAT5 in a plant with the same genetic background not transformed with the nucleic acid. In a preferred embodiment, the wildtype SINAT5 polypeptide is expressed in the transgenic plant at a level at of at least about two times the level of endogenous wildtype SINAT5 polypeptide. In another preferred embodiment, the wildtype SINAT5 polypeptide is expressed in the transgenic plant at a level of about at least five times greater than the level of endogenous wildtype SINAT5 polypeptide. In another preferred embodiment, the transgenic plant of the invention is grown from a transgenic plant cell of the invention and produces fewer lateral roots than the number of lateral roots produced in a plant not transformed with the wildtype SINAT5 and/or a longer main root stem than the main root stem in a plant not transformed with the nucleic acid.

The invention also provides vectors comprising a nucleic acid encoding a polypeptide selected from the group consisting of SEQ ID NO:1, SEQ ID NO:2 and SEQ ID NO:3, a nucleic acid as in SEQ ID NO:4, a cell transformed with a nucleic acid or vector of the invention and a plant transformed with a nucleic acid or vector of the invention. The nucleic acids of the invention can express said polypeptides both in vitro and in vivo using various techniques such as e.g., transduction, transfection or transformation of the nucleic acid or vector into a cell and inz vitro transcription and translation, as well known by a person of ordinary skill in the art. The nucleic acid can also be operably linked to inducible and/or repressible promoters and enhancers such that expression of the nucleic acid and encoded polypeptide can be regulated based on various physiological conditions and signals, as well known by a person of ordinary skill in the art.

In another embodiment, the invention provides a transgenic plant comprising a gene which overexpresses SINAT5 or a functionally equivalent polypeptide (meaning a polypeptide which is able to form dimers with SINAT5 or NAC1 and which is at least 70% identical with, preferably 80% identical with, more preferably 90% identical with, and most preferably at least 95% identical with the SINAT5 polypeptide of SEQ ID NO:1 and comprises the same activity as the wildtype SINAT5 polypeptide) which causes the transgenic plant to grow fewer lateral roots when compared to a nontransgenic plant that does not overexpress SINAT5.

In another embodiment, the invention provides a transgenic plant cell or plant comprising a mutant SINAT5 gene which encodes and expresses a mutant SINAT5 polypeptide or a functionally equivalent polypeptide (meaning a polypeptide which is able to form dimers with SINAT5 or a mutant SINAT5 or NAC1 and which is at least 70% identical with, preferably 80% identical with, more preferably 90% identical with, and most preferably at least 95% identical with SINAT5 and has an altered activity as compared to the wildtype SINAT5 polypeptide) which causes the transgenic plant to grow more lateral roots when compared to the number of lateral roots in a nontransgenic plant that does not express the mutant SINAT5 polypeptide. In one preferred embodiment, the mutant SINAT5 nucleic acid encodes a mutant polypeptide as in SEQ ID NO:2. In one embodiment, the mutant polypeptide is expressed at a level at least twice as great as the level of expression of wildtype SINAT5 in the cell or plant. In another preferred embodiment, the mutant SINAT5 polypeptide is expressed at levels at least five times greater than the level of expression of wildtype SINAT5 polypeptide in the cell or plant. In another embodiment, the invention provides a nucleic acid encoding and expressing a mutant SINAT5 polypeptide as in SEQ ID NO:3. In other embodiments, the invention provides a vector, a plant cell or a transgenic plant comprising said nucleic acid.

In another embodiment, the invention provides a method for growing a transgenic plant which is larger than a wildtype plant which comprises expressing a mutant SINAT5 gene in said plant during its growth cycle. The plant may be larger because it is heavier, because it has bigger leaves, because it has thicker stems, because it has more and/or shorter lateral roots. In a preferred embodiment, the transgenic plant will have more lateral roots than a wildtype plant. In a preferred embodiment, the mutant SINAT5 comprises a serine (ser) substituted for a cysteine (cys) at amino acid position 49 of the SINAT5 polypeptide (i.e., a “C49S” mutation) and the method comprises expressing the mutant SINAT5 at a level greater than the level of expression of wildtype SINAT5 in the transgenic plant. In a particularly preferred embodiment, the mutant SINAT5 gene comprises a nucleic acid encoding a polypeptide as in SEQ ID NO:2 or a polypeptide comprising the amino acid sequence set forth in SEQ ID NO:2. In another preferred embodiment, the transgenic plant produces more lateral roots and or a shorter main root length compared to a plant not expressing a polypeptide as in SEQ ID NO:2 or a polypeptide comprising the amino acid sequence set forth in SEQ ID NO:2.

In another embodiment, the invention provides a transgenic plant comprising a gene which expresses a mutant SINAT5 polypeptide or a functionally equivalent polypeptide, wherein said polypeptide comprises a nucleic acid encoding the mutant SINAT5 polypeptide of SEQ ID NO:2, which causes the plant to grow larger than a plant not expressing said mutant SINAT5 polypeptide. In a preferred embodiment, the transgenic plant expresses the mutant SINAT5 polypeptide at a level at least twice as great as the expression level of wildtype SINAT5 polypeptide in the plant. In another preferred embodiment, the transgenic plant expresses the mutant SINAT5 polypeptide at a level between 5 and 10 fold excess of the expression levels of wildtype SINAT5 polypeptide in the plant. In a preferred embodiment, the transgenic plant produces more lateral roots and or a shorter main root length compared to a plant not expressing the polypeptide.

### BRIEF DESCRIPTION OF THE FIGURES

FIG. 1a shows the sequence homology at the amino acid level between SINAT5 and other RING motif proteins.

FIG. 1b shows the results of in vivo dimerization studies with SINAT5.

FIG. 1c shows the results of in vitro dimerization studies with SINAT5.

FIG. 1d shows the results of SINAT5/NAC1 binding in vitro.

FIG. 1e shows the results of auxin induced expression patterns of SINAT5 and NAC1.

FIG. 1f shows the results of auxin induction of NAC1 and SINAT5 mRNA expression levels.

FIG. 1g shows the distribution of GFP-SINAT5 fusion protein in onion epidermal cells.

FIG. 2, a-d show the results of E3 ligase activity of wildtype and mutant SINAT5.

FIG. 3 shows the phenotypes of control plants and transgenic plants overexpressing SINAT5 or SINAT5 49S mutant genes.

FIG. 4, a-d show the results of Western and Northern blot analyses of SINAT5 and NAC1 protein and mRNA expression in transgenic plants.

### DETAILED DESCRIPTION OF THE INVENTION

A new Arabidopsis ubiquitin protein ligase, SINAT5, is described. This gene was originally isolated by using the transcription factor NAC1 of Arabidposis as a protein bait in a yeast two hybrid screening assay of an Arabidopsis thaliana cDNA library. SINAT5 was shown to have homology with C3HC4 RING motif proteins SINA from Drosophila and human SIAH. Subsequent analysis revealed that SINAT5 regulates NAC1 expression and thus development in plants, especially lateral root development and main root length. SINAT5 is shown to ubiquitylate NAC1 by functioning as an E3 ligase. SINAT5 also undergoes self-ubiquitylation Mutations in the RING structure of SINAT5 are shown to be incapable of self-ubiquitylation. In addition, one mutant in the RING structure of SINAT5, “C49S” (Substitution of Serine for Cysteine at amino acid 49 of the protein) inhibits self-ubiquitylation of wildtype SINAT5. Like NAC1, SINAT5 expression is induced by auxin but with slower kinetics.

As used herein, the singular form “a”, “an”, and “the” can include plural references unless the context clearly indicates otherwise. For example, a reference to “a” plant, vector, cell, nucleic acid and the like could include a plurality of plants, vectors, cells, nucleic acids and the like.

A polynucleotide or nucleic acid is said to “encode” a polypeptide if, in its native state or when manipulated by methods well known to those skilled in the art, it can be transcribed and/or translated to produce the mRNA for and/or the polypeptide or a fragment thereof. The anti-sense strand is the complement of such a nucleic acid, and the encoding sequence can be deduced therefrom.

An “isolated” or “substantially pure” nucleic acid (e.g., an RNA, DNA or a mixed polymer) or polypeptide is one which is substantially separated from other cellular components which naturally accompany a native human sequence or protein, e.g., ribosomes, polymerases, many other human genome sequences and proteins. The term embraces a nucleic acid sequence or protein that has been removed from its naturally occurring environment, and includes recombinant or cloned DNA isolates and chemically synthesized analogs or analogs biologically synthesized by heterologous systems. The present invention contemplates nucleic acids which comprise isolated SINAT5 nucleic acids.

The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. The polynucleotides of the invention may be isolated or substantially pure.

The present invention provides recombinant nucleic acids comprising the SINAT5 gene. The recombinant construct may be capable of replicating autonomously in a host cell. Alternatively, the recombinant construct may become integrated into the chromosomal DNA of the host cell. Such a recombinant polynucleotide comprises a polynucleotide of genomic, cDNA, semi-synthetic, or synthetic origin which, by virtue of its origin or manipulation, 1) is not associated with all or a portion of a polynucleotide with which it is associated in nature; 2) is linked to a polynucleotide other than that to which it is linked in nature; or 3) does not occur in nature.

Therefore, recombinant nucleic acids comprising sequences otherwise not naturally occurring are provided by this invention. Although the described sequences may be employed, it will often be altered, e.g., by deletion, substitution or insertion.

“Protein modifications or fragments” are provided by the present invention for wildtype and mutant SINAT5 polypeptides or fragments thereof which are substantially homologous to primary structural sequence but which include, e.g., in vivo or in vitro chemical and biochemical modifications or which incorporate unusual amino acids. Such modifications include, for example, acetylation, carboxylation, phosphorylation, glycosylation, ubiquitination, labeling, e.g., with radionuclides, and various enzymatic modifications, as will be readily appreciated by persons of ordinary skill in the art. A variety of methods for labeling polypeptides and of substituents or labels useful for such purposes are well known by persons of ordinary skill in the art, and include radioactive isotopes such as ³²P, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation.

Besides substantially full-length proteins, the present invention provides for biologically active fragments of the polypeptides. Significant biological activities include ligand-binding, immunological activity and other biological activities characteristic of proteins. The term “polypeptide” as used herein refers to both a full length protein and a portion of the protein as a polypeptide fragment.

A polypeptide “fragment,” “portion” or “segment” is a stretch of amino acid residues of at least about five to seven contiguous amino acids, often at least about seven to nine contiguous amino acids, typically at least about nine to 13 contiguous amino acids and, most preferably, at least about 20 to 30 or more contiguous amino acids.

The present invention also provides for fusion polypeptides, comprising SINAT5 polypeptides and fragments thereof and polypeptides or fragments of other proteins as known in the art. Homologous polypeptides may be fusions between two or more polypeptide sequences or between the sequences of SINAT5 and a related protein. Likewise, heterologous fusions may be constructed which would exhibit a combination of properties or activities of the derivative proteins. For example, ligand-binding or other domains may be “swapped” between different new fusion polypeptides or fragments. Such homologous or heterologous fusion polypeptides may display, for example, altered strength or specificity of binding and may include for example partners such as immunoglobulins, bacterial β-galactosidase, trpe, protein A, β-lactamase, alpha amylase, alcohol dehydrogenase and yeast alpha mating factor.

Fusion proteins will typically be made by either recombinant nucleic acid methods, as described below, or may be chemically synthesized. Techniques for the synthesis of polypeptides are well known by persons of ordinary skill in the art.

Other protein modifications include amino acid substitution. Substitutional variants typically contain the exchange of one amino acid for another at one or more sites within the protein, and may be designed to modulate one or more properties of the polypeptide, such as stability against proteolytic cleavage, without the loss of other functions or properties. Amino acid substitutions may be made on the basis of similarity in polarity, charge, solubility, hydrophobicity, hydrophilicity, and/or the amphipathic nature of the residues involved. Preferred substitutions are ones which are conservative, that is, one amino acid is replaced with one of similar shape and charge. Conservative substitutions are well known to persons of ordinary skill in the art and typically include, though not exclusively, substitutions within the following groups: glycine, alanine; valine, isoleucine, leucine; aspartic acid, glutamic acid; asparagine, glutamine; serine, threonine; lysine, arginine; and tyrosine, phenylalanine.

Certain amino acids may be substituted for other amino acids in a protein structure without appreciable loss of interactive binding capacity with structures such as, for example, antigen-binding regions of antibodies or binding sites on substrate molecules or binding sites on proteins interacting with an polypeptide. Since it is the interactive capacity and nature of a protein which defines that protein's biological functional activity, certain amino acid substitutions can be made in a protein sequence, and its underlying DNA coding sequence, and nevertheless obtain a protein with like properties. In making such changes, the hydropathic index of amino acids may be considered. The importance of the hydrophobic amino acid index in conferring interactive biological function on a protein is generally understood in the art. Alternatively, the substitution of like amino acids can be made effectively on the basis of hydrophilicity. The importance of hydrophilicity in conferring interactive biological function of a protein is generally understood in the art (See e.g. U.S. Pat. No. 4,554,101). The use of the hydrophobic index or hydrophilicity in designing polypeptides is further discussed in U.S. Pat. No. 5,691,198.

“Recombinant nucleic acid” is a nucleic acid which is not naturally occurring, or which is made by the artificial combination of two otherwise separated segments of sequence. This artificial combination is often accomplished by either chemical synthesis means, or by the artificial manipulation of isolated segments of nucleic acids, e.g., by genetic engineering techniques. This phrase is also meant to encompass a gene which is removed from its normal regulatory expression constraints, as in the case where a gene product is overexpressed due to the presence of multiple copies of the gene or up regulated promoter or enhancer signals, increased mRNA or protein half life and the like.

“Regulatory sequences” refers to those sequences which affect the expression of the gene (including transcription of the gene, and translation, splicing, stability or the like of the messenger RNA).

Large amounts of the polynucleotides of the present invention may be produced by a suitable host cell transformed with a nucleotide sequence encoding mutant or wildtype SINAT5 protein. Natural or synthetic polynucleotide fragments coding for the peptide or a desired fragment can be incorporated into recombinant polynucleotide constructs (vectors), usually DNA constructs, capable of introduction into and replication in a prokaryotic or eukaryotic cell. Usually the vectors will be suitable for replication in a unicellular host, such as yeast or bacteria, but may also be intended for introduction to (with and without integration within the genome) cultured mammalian or plant or other eukaryotic cell lines. The most commonly used prokaryotic hosts are strains of Escherichia coli, although other prokaryotes, such as Bacillus subtilis or Pseudomonas may also be used. Mammalian or other eukaryotic host cells, such as those of yeast, filamentous fungi, plant, insect, or amphibian or avian species, may also be useful for production of the proteins of the present invention. As is well known in the relevant art, regulating polynucleotide expression can result in regulation of polypeptides encoded by the polynucleotide.

Antibodies that specifically bind the polypeptides of the present invention are also contemplated. Using techniques well known to a person of ordinary skill in the art, the polypeptides can be used as antigen for inducing an antibody response in an animal and the antibodies generated can be screened for antibodies with specificity for a polypeptide of the present invention. The present invention also provides polyclonal and/or monoclonal antibodies and fragments thereof, and immunologic binding equivalents thereof, which are capable of specifically binding to the SINAT5 polypeptides and fragments thereof or to polynucleotide sequences from the SINAT5 region, particularly from the SINAT5 locus or a portion thereof. The term “antibody” is used both to refer to a homogeneous molecular entity, or a mixture such as a serum product made up of a plurality of different molecular entities. Polypeptides may be prepared synthetically in a peptide synthesizer and coupled to a carrier molecule (e.g., keyhole limpet hemocyanin) and injected over several months into rabbits. Rabbit sera is tested for immunoreactivity to the SINAT5 polypeptide or fragment. Monoclonal antibodies may be made by injecting mice with the protein polypeptides, fusion proteins or fragments thereof. Monoclonal antibodies will be screened by ELISA and tested for specific immunoreactivity with SINAT5 polypeptide. Antibodies can also comprise single chain or multimer recombinant antibodies or fragments thereof.

Vectors will include an appropriate promoter and other necessary vector sequences that are functional in the selected host. There may include, when appropriate, those naturally associated with the SINAT5 nucleic acid and protein expression and may include alternative or additional regulatory sequences operably linked to the recombinant SINAT5 gene in order to control SINAT5 gene expression, as well known in the art. Many useful vectors are known in the art and may be obtained from such vendors as Stratagene, New England BioLabs, Promega Biotech, and others. Promoters such as the trp, lac and phage promoters, tRNA promoters and glycolytic enzyme promoters may be used in prokaryotic hosts. Useful yeast promoters include promoter regions for metallothionein, 3-phosphoglycerate kinase or other glycolytic enzymes such as enolase or glyceraldehyde-3-phosphate dehydrogenase, enzymes responsible for maltose and galactose utilization, and others. “Operably linked” refers to a juxtaposition wherein the components so described are in a relationship permitting them to function in their intended manner. For instance, a promoter is operably linked to a coding sequence if the promoter affects its transcription or expression.

Expression and cloning vectors preferably contain a selectable marker gene. Typical marker genes encode proteins that a) confer resistance to antibiotics or other toxic substances, e.g. ampicillin, neomycin, methotrexate, etc.; b) complement auxotrophic deficiencies, or c) supply critical nutrients not available from complex media, e.g., the gene encoding D-alanine racemase for Bacilli. The choice of an appropriate proper selectable marker will depend on the host cell, and appropriate markers for different hosts are well known to persons of ordinary skill in the art.

The vectors containing the nucleic acids of interest can be transcribed in vitro, and the resulting RNA introduced into the host cell by well-known methods, e.g., by injection, or the vectors can be introduced directly into host cells by methods well known to persons of ordinary skill in the art, which vary depending on the type of cellular host, including electroporation; transfection employing calcium chloride, rubidium chloride, calcium phosphate, DEAE-dextran, or other substances; microprojectile bombardment; lipofection; infection (where the vector is an infectious agent, such as a retroviral genome); and other methods. The introduction of the polynucleotides into the host cell by any method known in the art, including, inter alia, those described above, will be referred to herein as “transformation.” The cells into which have been introduced nucleic acids described above are meant to also include the progeny of such cells.

Clones are selected by using markers, depending on the mode of the vector construction. The marker may be on the same or a different DNA molecule, preferably the same DNA molecule. In prokaryotic hosts, the transformant may be selected, e.g., by resistance to ampicillin, tetracycline or other antibiotics. Production of a particular product based on temperature sensitivity may also serve as an appropriate marker.

Prokaryotic or eukaryotic cells transformed with the polynucleotides of the present invention are useful not only for the production of the nucleic acids and polypeptides of the present invention, but also, for example, in studying the characteristics of SINAT5 polypeptides. Plant cells transformed with the polynucleotoides of the present invention are useful also for growing plants expressing the polynucleotides and polypeptides of the present invention. The nucleotides of the present invention can also be transformed into plants that have already undergone some growth. Plants not transformed with nucleic acids of the invention but expressing of endogeneous wildtype SINAT5 polypeptide produce on the average about 6.2 lateral roots per seedlings. Plants not transformed with nucleic acids of the invention but expressing of endogeneous wildtype SINTA5 polypeptide produce on the average main root length of about 3-4 cm. Plants overexpressing of SINAT5 produce less lateral root (about 3 lateral root per seeding, about 4.6 cm in main root length) than wildtype control. While plants overexpressing of mutated form of SINTA5 (SINTA5(CA49S)) produce more lateral roots (11 per seedlings) with main root length about 3 cm.

Antisense polynucleotide sequences are useful in preventing or diminishing the expression of the SINAT5 locus, as will be appreciated by those skilled in the art. For example, polynucleotide vectors containing all or a portion of the locus or other sequences from the region (particularly those flanking the locus) may be placed under the control of a promoter in an antisense orientation and introduced into a cell. Expression of such an antisense construct within a cell will interfere with transcription and/or translation of the SINAT5 gene.

General Methods

The methods employed in the following examples and in the practice of the present invention include those described below, other methods well known to persons of ordinary skill in the art and those described in Xie et al., Nature 419:167-171 (2002)²¹.

Yeast Two-Hybrid Screening. For yeast two-hybrid screens cDNAs were synthesized from 5 μg of poly(A) RNA isolated from plants at different growth stages using a commercial kit (Stratagene). The resulting double-stranded DNA's, containing EcoRI and XhoI ends (average size 1.2 kb), were ligated to the EcoRI/XhoI-digested pGAD-GH vector (Clontech) for 48 hr at 8° C. Following ligation, the cDNA library was used to transform E. coli DH10B (Gibco) by electroporation. The cDNA library contains ˜6×10⁶ primary transformants. Plasmids were isolated by standard methods. The yeast strain HF7c (MATa ura3-52 his3-200 ade2-101 lys2-801 trp1-901 leu2-3, 112 gal4-542 gal80-538 LYS2::GAL1UAS-GAL1TATA-HIS3 URA3::GAL4 17mers(x3)-CyC1TATA-LacZ, which contains the two reporter genes LacZ and HIS3, were used. Yeast cells were transformed sequentially with pGB-NAC1 (1-199), a plasmid containing sequences encoding the first 199 amino acids of NAC1 fused to the (Gal4 DNA-binding domain (BD;TRP1 marker) in the pGBT8 vector, and then with the Arabidopsis cDNA library constructed as described above. Details for screening have been described previously²².

Transgenic plants. A binary vector VIP96 (Kan^{R} in plants) carrying a 35S-6xMyc-NAC1 transgene was used to transform Arabidopsis thaliana Landsberg ecotype. Wildtype (WT) and homozygous Myc-NAC1 overexpressing plants were re-transformed with pBA002 (Basta resistance in plants)²³ carrying either 35S-SINAT5 or 35S-SINAT5(C49S). All mutations were created using the Quick-Change site-directed mutagenesis kit (Stratagene). For the SINAT5 promoter-GUS construct, a 2-kb fragment upstream from the ATG of SINAT5 was PCR-amplified and cloned into pBI101 (Clontech). Arabidopsis plants were transformed by the floral dip method²⁴ and transformants were selected on MS plates containing 50 μg/ml kanamycin and/or 10 μg/ml Basta. Details of plant growth conditions, auxin induction and GUS staining were described previously²⁰. For cellular localization of SINAT5, plasmid DNA containing a 35S-SINAT5-GFP construct was bombarded into onion peels using conditions described previously²⁰.

Preparation of proteins, in vitro binding and ubiquitylation assays. DNA encoding SINAT5 and the SINAT5 (C49S) mutant were cloned into pMal-c2 (New England Biolabs) and the fusion proteins were prepared, according to the manufacturer's instructions. [³⁵S] Methionine-labeled SINAT5 and NAC1 were generated by in vitro transcription and translation with wheat germ extracts using a T7/T3 coupled TnT kit (Promega). For in vitro binding, 2 μl of the translation mix was added to 200 μl binding buffer containing 50 mM HEPES (pH7.5), 1 mM EDTA, 150 mM NaCl, 10% Glycerol, 0.1% Tween 20 and 0.5 mM DTT, and the mixture incubated at room temperature for 1 hr. After the incubation the beads containing amylose resin were washed three times with the washing buffer (50 mM Tris-HCl, pH7.5, 150 mM NaCl and 0.2% NP-40). ³²P-labeled Ub was prepared using recombinant GST-Ub as described ^{9,1}Unlabeled Ub (10 μg/per reaction) was purchased from Sigma. Preparation of wheat E1 (cDNA clone kindly provided by Dr. R Vierstra) and UbcH5B (E2) were previously reported^{9,11}. For in vitro ubiquitylation, MBP-SINAT5 and/or C49S mutant (500 ng) were immobililzed on amylose resin beads. Ubiquitylation assays were performed by adding 20 ng each of recombinant wheat E1, UbcH5B, and 2×10⁴ cpm of ³²P-labeled Ub in ubiquitylation buffer containing 50 mM Tris, pH 7.4, 2 mM ATP, 5 mM MgCl², 2 mM DTT. The reaction mix (30 μl) was incubated for 1.5 h at 30° C. with agitation in an Eppendorf Thermomixer. Following the reaction, samples were heated to 95° C. in a SDS-PAGE sample buffer containing β-mercaptoethanol prior to electrophoresis on 10% SDS gels.

Western and Northern blot analyses. Antibody to SINAT5 was raised in rabbits using as an antigen the synthetic 14-mer peptide TDSIDSVIDDDEIH (SEQ ID NO:5) corresponding to amino acid residues 3 to 18 of SINAT5 polypeptide (SEQ ID NO:1). Antibodies to c-Myc (SC40 and SC40-AC) and actin (SC1616) from Santa Cruz Biotechnology were used for immunoprecipitation and/or Western blotting. Each lane contained 10 or 20 μg protein. RNA extraction was performed by RNeasyR Plant Mini kit (Qiagene). Northern blot analysis was described previously²⁰, using as probes a NAC1 cDNA fragment encoding the NAC1 C-terminal region (amino acid 200 to 324) and a SINAT5-specific EcoRI-NdeI fragment. The 18S rRNA was used as a loading control. Each lane contained 10 μg RNA.

The present invention is described by reference to the following Examples, which are offered by way of illustration and are not intended to limit the invention in any manner. Standard techniques well known by persons of ordinary skill in the art and/or the techniques specifically described herein were utilized.

### EXAMPLE 1

Isolation of the SINAT5 gene and determination of the SINAT5 nucleic acid and amino acid sequence. Using techniques well known in the art, yeast two hybrid assays using NAC1 as a bait were performed on cDNA libraries from Arabidopspis thaliania. Positive clones were identified and the nucleotide sequences of the positive clones were determined using methods well known to persons of ordinary skill in the art. Amino acid sequence homology studies were performed on the positive clones to identify potential clones of interest. One NAC1-interacting protein identified with the yeast two hybrid system shows extensive sequence homology with the C3HC4 RING motif proteins Drosophila SINA²⁵ and human SIAH²⁶. Because the gene encoding this protein is located on chromosome 5, we have designated this protein as SINAT (ie., SINA of Arabidopsis thaliana) 5 (Genbank Accession No: AF480944). The amino acid sequence of SINAT5 is shown in SEQ ID NO:1 and the nucleotide sequence which encodes SEQ ID NO:1 is shown in SEQ ID NO:4.

### EXAMPLE 2

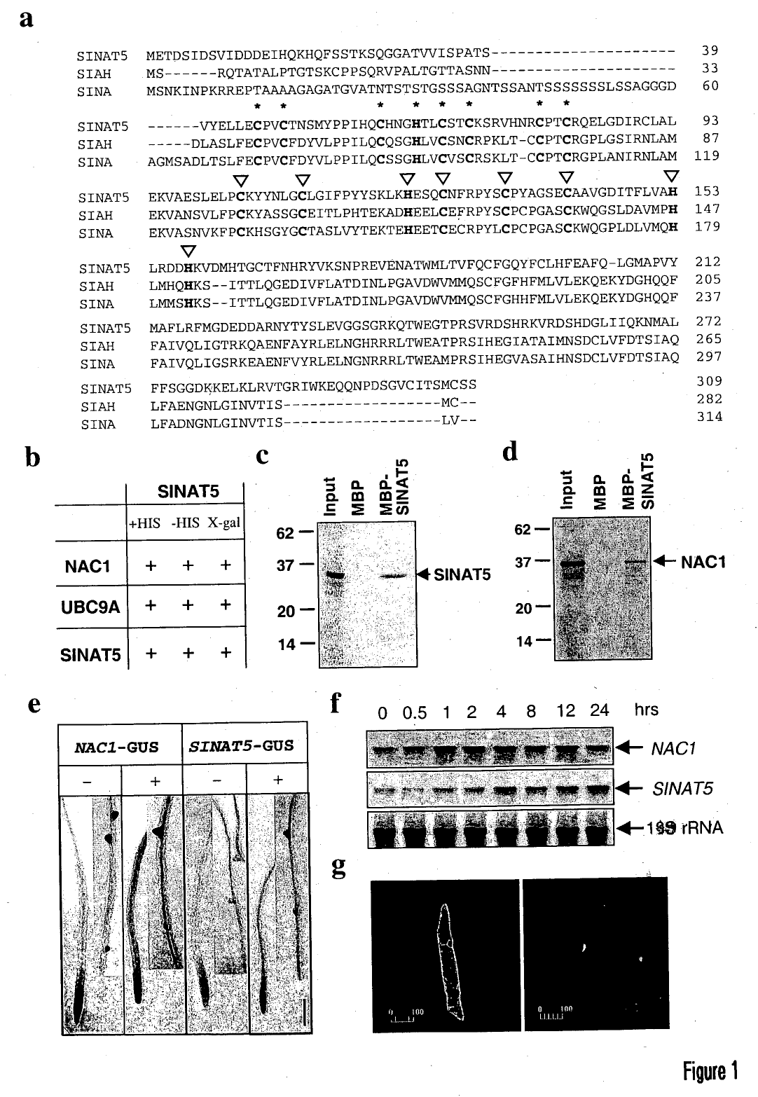

Amino acid sequence homology of SINAT5. Referring now to FIG. 1a, there are shown sequence comparisons between the SINAT5 protein and other SIN homologs. SINAT5 shares extensive sequence homology with the SINA/SIAH family^{25,26}. The identities between SINAT5 and SINA (M38384) and SIAH (U76247) are 33% and 36%, respectively. The comparisons were done using the NCBI “tblast” program.

### EXAMPLE 3

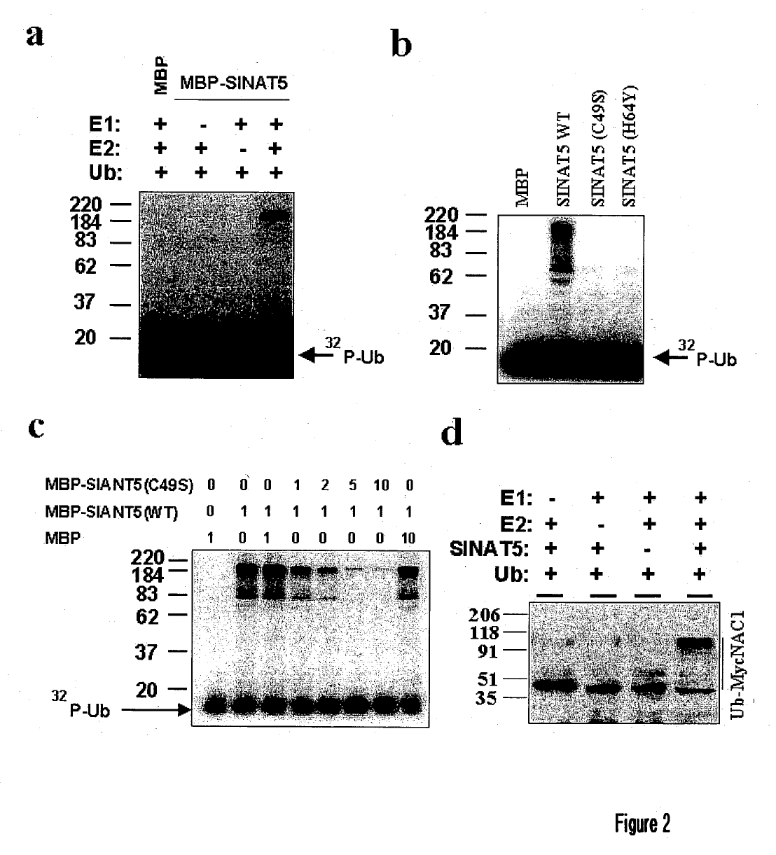

Regulation of auxin-signaling pathway is also regulated by ubiquitin-mediated proteolysis. Referring now to FIG. 2, there are shown the results of E3 ubiquitin-mediated ligase activity of WT and mutant (SINAT5), which represents the potential proteolytic activity of thje mutant and WT proteins. Ubiquitylation (E3 activity) was performed as described in the General Methods and is represented by the presence of the multimer at approximately 220 kd which represent self-ubiquitylated SINAT 5 (FIGS. 2a-c) or ubiquitylated NAC1 protein (2d). In FIG. 2a, MBP-SINAT5 was assayed for E3 activity in the presence of E1, E2 and 32p-Ub as indicated. FIG. 2b demonstrates that MBP-SINAT5 E3 activity is dependent on its RING finger. MBP-SINAT5 and mutants were assayed for self-ubiquitylation. FIG. 2c demonstrates that mutant SINAT5 (C49S) blocked the E3 activity of wildtype SINAT5. Numbers indicate the relative amount of proteins present in the various ubiquitylation reactions. Number 1 equals 100 ng of MBP or MBP-SINAT. d, Ubiquitylation of NAC1 by SINAT5. Referring now to FIG. 2d, Myc-NAC1 immunoprecipitated from transgenic plant was assayed for in vitro ubiquitylation. Samples were processed for Western blot analysis using anti-myc antibody or anti SINAT5 antibody. In the presence of ubiquitin (Ub), E1 and E2, purified MBP-SINAT5 was able to perform self-ubiquitylation whereas purified MBP alone was inactive (FIG. 2a). As negative controls, we constructed two mutations (C49S and H64Y) that disrupt the RING domain (FIG. 1a) and both mutants were totally incapable of self-ubiquitylation (FIG. 2b) suggesting that an intact RING finger is needed for E3 activity. Moreover, the C49S mutant protein was able to inhibit the self-ubiquitylation activity of the WT SINAT5 when present at a 5-10 fold excess (FIG. 2c). A ten-fold increase in E2 amounts did not reduce the inhibition (data not shown) indicating that the E2 was not simply sequestered by the excess SINAT5 (C49S) mutant protein added. These results show that the SINAT5 E3 activity requires dimerization and that the inactive SINAT5 (C49S) mutant can act as a dominant negative, probably by forming inactive dimers with WT SINAT5.

The interaction between SINAT5, an E3 ligase, and NAC1 suggests that the former may ubiquitylate the latter in vivo and target it for proteasomal degradation. To provide direct evidence that NAC1 is a substrate of SINAT5 ubiquitylation experiments using myc-NAC1 immunopurified from transgenic plants overexpressing the tagged protein were performed. FIG. 2d shows that myc-NAC1 can be ubiquitylated by SINAT5 and this reaction is clearly dependent on E1, E2, and SINAT5 as an E3 ligase.

### EXAMPLE 4

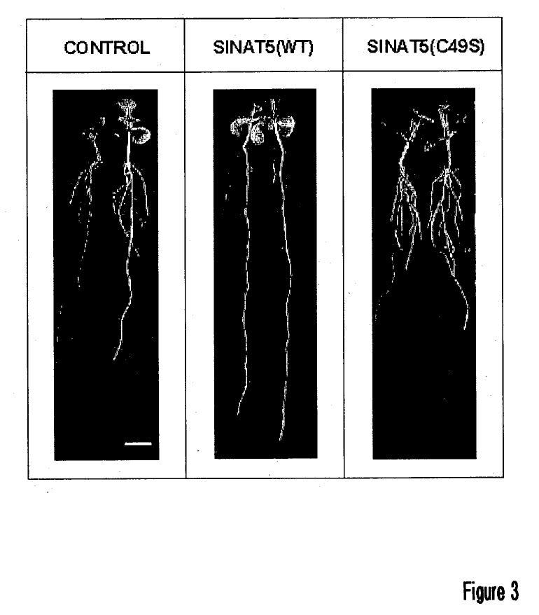

Overexpression of wildtype and C49S mutant SINAT5. FIG. 3 shows the phenotypes of transgenic plants of the invention. Seedlings of WT Landsberg ecotype (CONTROL), transgenic lines carrying a 35S-SINAT5 transgene [SINAT5 (WT)] and transgenic plants carrying a 35S-SINAT5(C49S) trans gene [SINAT5 (C49S)] were grown vertically on MS agar medium containing 2% sucrose. Representative plants were photographed after 10 days. Size bar, 0.5 cm. The average number of lateral roots per seedling (+SD) and the length of main root per seedling (in parenthesis) were determined for 30 seedlings each. CONTROL, 6.2±0.3 (3.4±0.4 cm); SINAT5 (WT), 2.7±0.4 (4.6±0.4 cm) and SINAT5 (C49S), 10.9±0.2 (2.5±0.3 cm).

Since overexpression of NAC1 produced more lateral roots whereas under-expression of NAC1 produced fewer lateral roots, if SINAT5 ubiquitylates NAC1 in vivo, thereby promoting its degradation, overexpression of WT SINAT5 would be expected to reduce lateral root numbers. Conversely, overexpression of the dominant-negative C49S mutant would be expected to increase lateral root numbers. These results were indeed obtained in transgenic experiments (FIG. 3). Moreover, the main root length of SINAT5 plants was longer than that of WT plants whereas the converse was true for C49S plants. This effect of SINAT5 is similar to that observed in the auxin-response mutant axr-1¹. In a separate demonstration, 10 day-old seedlings (n=30) were transferred from MS medium to the same medium with 2 μM 1-NAA for 48 hrs. The number of lateral roots per cm of main root was: vector control, 13.4 (±0.4), SINAT5-overexpressing plants, 7.2 (±0.5), and C49S-overexpressing plants, 19.7 (±0.5). These results show that SINAT5 inhibits auxin-induced lateral root development whilst the C49S mutant promotes the same process.

### EXAMPLE 5

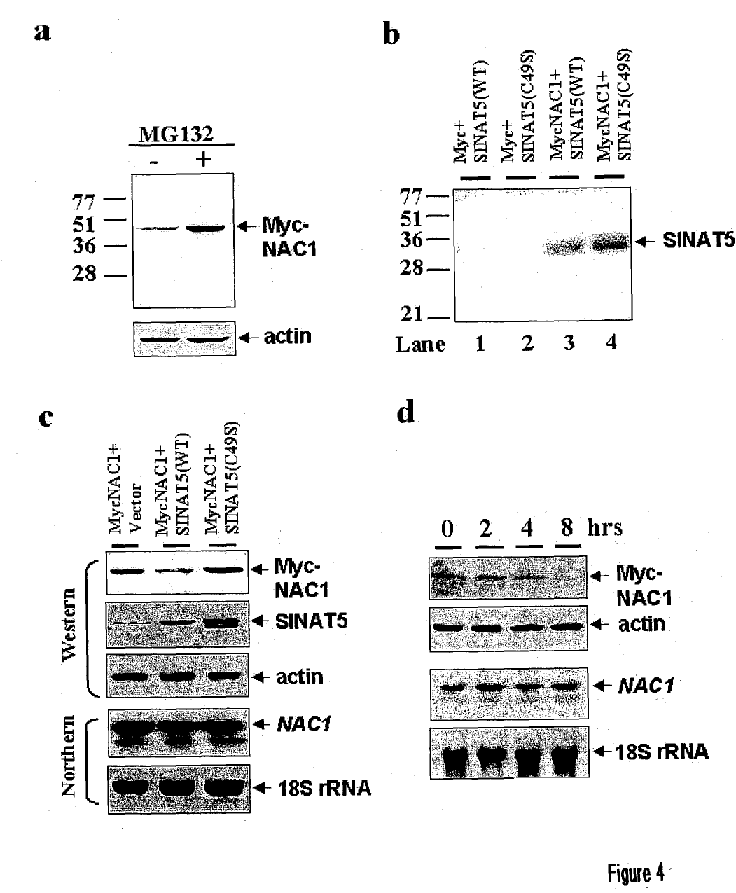

SINAT5 affect on expression of NAC1 in roots. Referring now to FIG. 4, there is shown the relationship between SINAT5 and NAC1 expression levels in transgenic plants. FIG. 4a demonstrates that NAC1 expression level is increased by MG132 treatment. Root cultures from a myc-NAC1 transgenic line were treated with 50 μM of proteasome inhibitor MG 132 (+) or DMSO (−) for 3 hr. Protein extracts were processed for Western blot analysis. FIG. 4b shows that SINAT5 interacts with NAC1 in vivo. Myc-tagged proteins from roots of double transgenic plants were immunoprecipitated with antibodies specific for Myc and processed for Western blot analysis with antibodies specific for SINAT5. FIG. 4c shows the relative expression levels of SINAT5 and NAC1 in transgenic roots. Total RNAs from the same transgenic lines were analyzed by Northern blot. FIG. 4d shows that Auxin promotes Myc-NAC1 degradation. Root cultures from Myc-NAC1 transgenic line were treated with 2 μM 1-NAA. Upper two panels, Western analysis; lower two panels, Northem analysis.

The expression level of NAC1 in roots is low but can be increased considerably by the proteasome inhibitor MG 132 (FIG. 4a) suggesting that the low expression level is likely due to continuous ubiquitin-mediated proteolysis. Pull-down assays using extracts prepared from transgenic plants demonstrated that both WT SINAT5 and the C49S mutant were able to interact with NAC1 in plant cells (FIG. 4b) as demonstrated by the appearance of positive bands only in the double transgenic plants and not in control. Consistent with the posttranslational regulation of NAC1 by SINAT5 in vivo, overexpression of WT SINAT5 resulted in a reduced NAC1 levels in roots whereas overexpression of the C49S mutant led to higher NAC1 levels (FIG. 4c). In these transgenic plants, the NAC1 transcript levels remained at about the same level. Treatment of roots with auxin caused a reduction in NAC1 levels without any apparent effect on the expression level of the 35S-Myc-NAC1 transcript levels (FIG. 4d), indicating that the hormone triggers posttranslational proteolysis of the transcription activator.

Both SINAT5 and NAC1 expression are induced by auxin although the former displays a slower kinetics. This difference in the induction kinetics may explain the auxin-induced degradation of NAC1, which becomes apparent at 8 hr after hormone treatment (FIGS. 1f and 4d).

### EXAMPLE 6

Determination of SINAT5-binding proteins in Arabidopsis. Using SINAT5 as a bait, we found that it interacted with Arabidopsis AtUBC9A (AF480945; homologous to yeast Ubc 4/5p and human Ube H5 family members) ubiquitin (data not shown), as well as SINAT5 itself (FIG. 1b; +His is control and His− shows results of assay). FIG. 1b shows the results of two hybrid screening in yeast where SINAT5 was used as the “bait” protein. This figure shows that SINAT5 interacted in vivo with Arabidopsis UBC9A, itself and NAC1. This figure reflects a positive (+) interaction as expression of cell growth and β-galactosidase activity. Using similar techniques, it was also determined that the C-terminal region is also responsible for SINAT5 dimerization consistent with recent results reported for SIAH^{27,28}. The dimerization of SINAT5 and the interaction of the latter with NAC1 were verified by in vitro pull-down assays. Referring now to FIG. 1c, there are shown the results of dimerization of SINAT5 in vitro. Recombinant MBP or MBP-SINAT5 was mixed with in vitro translated SINAT5 as described in the General Methods. SINAT5 was labeled with 35S. Referring now to FIG. 1d, there are shown the results of SINAT5 binding to NAC1 in vitro. The methods for this assay were the same as in FIG. 1c except that in vitro translated NAC1 was used in place of in vitro translated SINAT5. NAC1 protein was labeled with 35S.

### EXAMPLE 7

Transcriptional Expression profile of SINAT5. The expression profile of SINAT5 was determined by generating transgenic Arabidopsis plants carrying a SINAT5 promoter-beta glucuronidase (GUS) fusion gene. Two week-old transgenic seedlings were treated with (+) or without (−)2 μM 1-NAA for 6 hours before being processed for GUS staining. The SINAT5-GUS fusion product was expressed at a low level in the vascular tissues of mature roots. However, upon treatment with auxin expression, the fusion product was also detected in lateral root initials and the elongation zone of the main root (FIG. 1e). This root expression pattern is very similar to that of NAC1 upon auxin induction⁹, demonstrating that SINAT5 and NAC1 function in the same cell types. FIG. 1f demonstrates that Auxin induces expression levels of both NAC1 and SINAT5. Cultured roots from wild type seedlings²⁰ were treated with 2 μM 1-NAA and RNA expression levels of NAC1 and SINAT5 gene transcripts at different times were analyzed. Northern blots of RNA were screened with probes specific for SINAT5 gene, NAC1 gene or 18s ribosomal RNA. Like NAC1, the expression level of SINAT5 was induced by auxin but with a slower kinetics (FIG. 1f).

### EXAMPLE 8

Sub-cellular localization of SINAT5 protein expression. To investigate the subcellular localization of SINAT5, we constructed a green fluorescent protein/SINAT5 fusion gene (GFP-SINAT5) under the control of a CaMV 35S promoter. Transient expression in onion epidermal cells demonstrated that GFP (Green Fluorescent Protein) was distributed in both the cytosol and the nucleus whereas GFP-SINAT5 was localized predominantly in the nucleus, as reported previously with NAC1²⁰ (FIG. 1g). Cells were analyzed by confocal microscopy after 16 hours of incubation.

Discussion

These results demonstrate that SINAT5, an Arabidopsis homolog of the RING motif protein SINA, can act as an ubiquitin protein ligase (E3) to ubiquitylate NAC1 and that the activity is abolished by mutations in the RING motif of SINAT5. Consistent with its role as an E3 for NAC1, transgenic plants overexpressing SINAT5 produce fewer lateral roots whereas those overexpressing the dominant negative SINAT5 (C49S) mutant develop more, in the absence as well as the presence of auxin. The lateral root phenotype correlates with NAC1 expression levels in vivo. Moreover, the low NAC1 expression levels in roots can be elevated by treatment with a proteasome inhibitor. These results provide evidence that SINAT5 targets NAC1 for ubiquitin-mediated proteolysis to down regulate auxin signals in plant cells.

Several lines of evidence support the notion that SINAT5 targets NAC1 for post-translational degradation: (1) NAC1, expression levels in vivo can be elevated by the proteosome inhibitor MG 132, (2) NAC1 can be ubiquitylated by SINAT5 in vitro, (3) NAC1 and SINAT5 are expressed in the same cell types and interact in vivo, and (4) Overexpression of WT SINAT5 in transgenic plants leads to a decrease in NAC1 levels in roots and a concomitant reduction of lateral root numbers whereas the opposite is found in transgenic plants overexpressing the dominant negative C49S mutant. The opposing effects of SINAT5 and the C49S mutant are also observed when lateral root development is induced by auxin thereby implicating the E3 ligase in this hormonal response. These findings provide additional insights into the function of the family as E3 ligase. Without being bound by theory, the capacity of a RING mutant SINAT5 to function as a dominant negative in in vitro ubiquitylation suggests that RING-independent dimerization of this protein family is of functional importance in its E3 activity. Dimerization of RING finger containing proteins as a means of activating E3 activity is in accord with results obtained with BRCA1-BARD1 heterodimers²⁹.

In contrast to TIR1, which activates auxin signaling by protein degradation⁶ SINAT5 attenuates the signal by targeting NAC1 for degradation, thereby resetting the transduction cascade. The transcriptional activation of both NAC1²⁰ and SINAT5 (FIG. 1e) by auxin indicates the importance of antagonistic transcriptional as well as posttranscriptional mechanisms in fine-tuning NAC1 -regualated aspects of auxin signaling.

Bibliography

1. del Pozo, J. C., Timpte, C., Tan, S., Callis, J. & Estelle, M. The ubiquitin-related protein RUB1 and auxin response in Arabidopsis. Science 280,1760-1763 (1998).

2. Ruegger, M., Dewey, E., Gray, W. M., Hobbie, L., Turner, J. & Estelle M. The TIR1 protein of Arabidopsis functions in auxin response and is related to human SKP2 and yeast grrlp. Genes Dev. 12, 198-207 (1998).

3. Rogg, L. E., Lasswell, J. & Bartel, B. A gain-of-function mutation in IAA28 suppresses lateral root development. Plant Cell. 13, 465-480 (2001).

4. Ouellet, F., Overvoorde, P. J. & Theologis, A. IAA17/AXR3: biochemical insight into an auxin mutant phenotype. Plant Cell 13, 829-841 (2001).

5. Gray, W. M. et al. Identification of an SCF ubiquitin-ligase complex required for auxin response in Arabidopsis thaliana. Genes Dev. 13, 1678-1691 (1999).

6. Gray, W. M., Kepinski, S., Rouse D., Leyser O. & Estelle, M. Auxin regulates SCF(TIR1)-dependent degradation of AUX/IAA proteins. Nature 414, 271-276 (2001).

7. Ramos, J .A., Zenser, N., Leyser, O. & Callis, J. Rapid (degradation of auxin/indoleacetic acid proteins requires conserved amino acids of domain II and is proteasome dependent. Plant Cell. 13, 2349-2360 (2001).

8. Zenser N., Ellsmore A., Leasure C., & Callis J. Auxin modulates the degradation rate of Aux/IAA proteins. Proc. Natl. Acad. Sci. USA 98, 11795-11800 (2001).

9. Lorick, K. L., Jensen, J. P., Fang, S., Ong, A. M., Hatakeyama, S. & Weissman, A. M. RING fingers mediate ubiquitin-conjugating enzyme (E2)-dependent ubiquitination. Proc. Natl. Acad. Sci. USA 96,11364-11369 (1999).

10. Joazeiro, C. A. et al. The tyrosine kinase negative regulator c-Cbl-Ubch7 as a Ring-type, E2 dependent ubiquitin-protein ligase. Science 286, 309-312 (1999).

11. Fang, S., Jensen, J. P., Ludwig, R. L., Vousden, K. H. & Weissman, A. M. Mdm2 is a RING finger-dependent ubiquitin protein ligase for itself and p53. J. Biol. Chem. 275, 8945- 8951 (2000).

12. Li, S., Li, Y., Carthew, R. W. & Lai, Z. C. Photoreceptor cell differentiation requires regulated proteolysis of the transcriptional repressor Tramtrack. Cell. 90 ,469-478 (1997).

13. Hu, G. et al. Mammalian homologs of seven in absentia regulate DCC via the ubiquitin-proteasome pathway. Genes Dev. 11,2701-2747 (1997).

14. Nie, J., McGill, M. A., Dermer, M., Dho, S. E., Wolting, C. D. & McGlade, C. J. LNX functions as a RING type E3 ubiquitin ligase that targets the cell fate determinant Numb for ubiquitin-dependent degradation. EMBO J. 21, 93-102 (2002).

15. Susini, L. et al. Siah-1 binds and regulates the function of Numb. Proc. Natl. Acad. Sci. USA 98 15067-15072 (2001).

16. Matsuzawa, S. &Reed, J. C. Siah-1, SIP, and Ebi collaborate in a novel pathway for ε-catenin degradation linked to p53 responses. Mol. Cell 7 915.926 (2001).

17. Boehm, J., He, Y., Greiner, A., Staudt, L., Wirth, T. Regulation of BOB.1/OBF.1 Stability by SIAH. EMBO J. 20 4153-4162 (2001).

18. Liu, J. et al. Siah-1 mediates a novel B-catenin degradation pathway linking p53 to the adenomatous polyposis coli protein. Mol. Cell 7 927-936 (20(11).

19.Tiedt, R., Bartholdy, B. A., Matthias, G., Newell, J. W., Matthias, P. The RING finger protein Siah-1 regulates the level of the transcriptional coactivator OBF-1 EMBO J. 20 4143-4152 (2001).

20.Xie, Q., Frugis, G., Colgan, D. & Chua, N. H. Arabidopsis NAC1 transduces auxin signal downstream of TIR1 to promote lateral root development. Genes Dev. 14, 3024-3036 (2000).

21. Xie, Q., Guo, H. S., Dallman, G., Fang, S., Weissman, A. and Chua, N. H. Nature 419:167-171 (2002).

22. Xie, Q., Sanz-Burgos, A. P., Guo, H., Garcia, J. A. and Gutierrez, C. GRAB proteins, novel members of the NAC domain family, isolated by their interaction with a geminivirus protein. Plant Mol Biol. 39, 647-56 (1999).

23. Kost, B., Spielhofer, P. & Chua, N. H. A GFP-mouse talin fusion protein labels plant actin filaments in vivo and visualizes the actin cytoskeleton in growing pollen tubes. Plant J. 16, 393-401 (1998).

24. Clough, S. J. & Bent, A. F. Floral dip: a simplified method for Agrobacterium-mediated transformation of Arabidopsis thaliana. Plant J. 16, 735-743 (1998).

25. Carthew, R. W. & Rubin, G. M. Seven in absentia, a gene required for specification of R7 cell fate in the Drosophila eye. Cell 63, 561-577 (1990).

26. Nemani, M. et al. Activation of the human homologue of the Drosophila sina gene in apoptosis and tumor suppression. Proc. Natl. Acad. Sci. USA 93, 9039-9342 (1996).

27. Hu, G. & Fearon, E. R. Siah-1 N-terminal RING domain is required for proteolysis function, and C-terminal sequences regulate oligomerization and binding to target proteins. Mol. Cell. Biol. 19, 724-732 (1999).

28. Polekhina, G. et al. Siah ubiquitin ligase is structurally related to TRAF and modulates TNF-signaling. Nat. Struct.Biol. 9, 68 -75 (2002).

29. Hashizume, R. et al. A RING Heterodimer BRCA1-BARD1 Is a Ubiquitin Ligase Inactivated by a Breast Cancer-derived Mutation. J. Biol. Chem. 276 14537-14540 (2001).

What is claimed is:

1. A transgenic plant cell comprising a recombinant nucleic acid encoding the polypeptide of SEQ ID NO:1, wherein the polypeptide is overexpressed in said cell when compared to a wildlype expressing the polypeptide.

2. A transgenic plant grown from the cell of claim 1, wherein the nucleic acid encoding the polypeptide is overexpressed during the growth of the plant and wherein the polypeptide is overexpressed during the growth of the plant as compared to a the level of expression of the polypeptide in a plant not overexpressing the polypeptide.

3. The transgenic plant of claim 2, wherein the plant has fewer lateral roots as compared to the number of lateral roots in a plant not overexpressing the polypeptide.

4. The transgenic plant of claim 3 wherein the plant produces less than 6 lateral roots per seedling.

5. The transgenic plant of claim 2, wherein the main root length of the transgenic plant is longer than the main root length in a plant not overexpressing the polypeptide.

6. The transgenic plant of claim 5 wherein the main root length is at least 4 cm in length.

7. The transgenic plant of claim 2, wherein the expression of the nucleic acid encoding the polypeptide is controlled by a regulatable plant promoter.

8. The transgenic plant of claim 2 which is Arabadopsis.

9. The transgenic plant of claim 8 which is Arabadopsis thaliana.

10. The transgenic plant of claim 2 wherein the polypeptide is expressed at a level of at least two times the level of expression of the polypeptide in a wildtype plant expressing the polypeptide.

11. The transgenic plant of claim 2 wherein the polypeptide is expressed at a level of at least two times the level of expression of the polypeptide in a wildtype plant expressing the polypeptide.

12. An isolated nucleic acid encoding the polypeptide of SEQ ID NO:1.

13. The isolated nucleic acid of claim 12 which comprises SEQ ID NO:4 or a nucleic acid which is complementary to SEQ ID NO:4.

14. A plant vector comprising the nucleic acid of claim 12.

15. A plant cell transformed with the nucleic acid of claim 14.

16. A transgenic plant cell comprising a nucleic acid encoding the polypeptide of SEQ ID NO:2, wherein the polypeptide is expressed in said cell.

17. The transgenic plant cell of claim 16 wherein the polypeptide of SEQ ID NO:2 is expressed at a level at least two times greater than the level of expression of wildtype SINAT5 polypeptide expressed in the cell.

18. A transgenic plant grown from the cell of claim 17, wherein the polypeptide of SEQ ID NO:2 is expressed at a level at least two times greater than the level of expression of wildtype SINAT5 polypeptide during the growth of the plant.

19. The transgenic plant of claim 18, wherein the plant has more lateral roots as compared to the number of lateral roots in a plant not expressing the polypeptide of SEQ ID NO:2 at a level greater than the level of expression of wildtype SINAT5 polypeptide.

20. The transgenic plant of claim 19 wherein the plant has more than 6 lateral roots per seedling.

21. The transgenic plant of claim 18, wherein the main root length of the transgenic plant is shorter than the main root length of a plant not expressing the polypeptide at a level greater than the level of expression of wildtype SINAT5 polypeptide.

22. The transgenic plant of claim 21 wherein the main root length of the plant is less than 3 cm.

23. The transgenic plant of claim 18, wherein the expression of the nucleic acid encoding the polypeptide of SEQ ID NO:2 is controlled by a regulatable plant promoter.

24. The transgenic plant of claim 18 which is Arabadopsis.

25. The transgenic plant of claim 24 which is Arabadopsis thaliana.

26. The transgenic plant cell of claim 16 wherein the polypeptide of SEQ ID NO:2 is expressed at a level at least five times greater than the level of expression of wildtype SINAT5 polypeptide expressed in the cell.

27. A transgenic plant grown from the cell of claim 26, wherein the polypeptide of SEQ ID NO:2 is expressed at a level at least five times greater than the level of expression of wildtype SINAT5 polypeptide during the growth of the plant.

28. The transgenic plant of claim 27, wherein the plant has more lateral roots as compared to the number of lateral roots in a plant not expressing the polypeptide of SEQ ID NO:2 at a level greater than the level of expression of wildtype SINAT5 polypeptide.

29. The transgenic plant of claim 28, wherein the main root length of the transgenic plant is shorter than the main root length of a plant not expressing the polypeptide at a level greater than the level of expression of wildtype SINAT5 polypeptide.

30. The transgenic plant of claim 29 wherein the main root length is less than 3 cm.

31. The transgenic plant of claim 24, wherein the expression of the nucleic acid encoding the polypeptide of SEQ ID NO:2 is controlled by a regulatable plant promoter.

32. The transgenic plant of claim 27 which is Arabadopsis.

33. The transgenic plant of claim 27 which is Arabadopsis thaliana.

34. An isolated nucleic acid encoding the polypeptide of SEQ ID NO:2.

35. A plant vector comprising the nucleic acid of claim 34.

36. A plant cell transformed with the nucleic acid of claim 35.

37. A transgenic plant cell comprising a nucleic acid encoding the polypeptide of SEQ ID NO:3, wherein the polypeptide is expressed in the cell.

38. A transgenic plant grown from the cell of claim 37, wherein the polypeptide is expressed during the growth of the plant.

39. The transgenic plant of claim 38, wherein the expression of the nucleic acid encoding the polypeptide of SEQ ID NO:2 is controlled by a regulatable plant promoter.

40. An isolated nucleic acid encoding the polypeptide of SEQ ID NO:3.

41. A plant vector comprising the nucleic acid of claim 40.

42. A plant cell transformed with the nucleic acid of claim 40.

43. A method of growing a transgenic plant comprising transforming a plant with a nucleic acid encoding the polypeptide of SEQ ID NO:1, wherein the polypeptide is expressed during the growth of the plant at a level greater than the level of expression in a plant not transformed with the nucleic acid, wherein the main root length of the plant is longer than the main root length of a plant not transformed with the nucleic acid.

44. The method of claim 43 which produces a transgenic plant having a main root length of at least 4 cm.

45. A method of growing a transgenic plant comprising transforming a plant with a nucleic acid encoding the polypeptide of SEQ ID NO:1, wherein the polypeptide is expressed during the growth of the plant at a level greater than the level of expression in a plant not transformed with the nucleic acid, wherein the transgenic plant produces fewer lateral roots than the number of lateral roots in a plant not transformed with the nucleic acid.

46. The method of claim 45 which produces a transgenic plant having less than 4 lateral roots per seedling.

47. The method of claim 45 wherein the polypeptide of SEQ ID NO:1 is expressed at a level at least two times greater than the level of expression of wildtype SINAT5 in the plant.

48. The method of claim 45 wherein the polypeptide of SEQ ID NO:1 is expressed at a level at least five times greater than the level of expression of wildtype SINAT5 in the plant.

49. A method of growing a transgenic plant comprising transforming a plant with a nucleic acid encoding the polypeptide of SEQ ID NO:2, wherein the polypeptide is expressed during the growth of the plant, wherein the main root length of the plant is shorter than the main root length of a plant not expressing the polypeptide.

50. The method of claim 49 which produces a plant with a main root length of less than 3 cm.

51. The method of claim 49 wherein the polypeptide of SEQ ID NO:2 is expressed at a level at least two times greater than the level of expression of wildtype SINAT5 in the plant.

52. The method of claim 49 wherein the polypeptide of SEQ ID NO:2 is expressed at a level at least five times greater than the level of expression of wildtype SINAT5 in the plant.

53. A method of growing a transgenic plant comprising transforming a plant with a nucleic acid encoding the polypeptide of SEQ ID NO:2, wherein the polypeptide is expressed during the growth of the plant, wherein the transgenic plant produces more lateral roots compared to the number of lateral roots in a plant not expressing the polypeptide.

54. The method of claim 53 which produces a plant having more than 6 lateral roots per seedling.

55. The method of claim 53 wherein the polypeptide of SEQ ID NO:2 is expressed at a level at least two times greater than the level of expression of wildtype SINAT5 in the plant.

56. The method of claim 53 wherein the polypeptide of SEQ ID NO:2 is expressed at a level at least five times greater than the level of expression of wildtype SINAT5 in the plant.

57. An antibody that specifically binds a SINAT5 polypeptide comprising an amino acid sequence set forth in SEQ ID NO:1, SEQ ID NO:2 or SEQ ID NO:3.

58. The antibody of claim 57 that is a monoclonal antibody.

59. The antibody of clam 57 that specifically binds a SINAT5 polypeptide comprising the amino acid sequence set forth in SEQ ID NO:1.

60. The antibody of clam 57 that specifically binds a SINAT5 polypeptide comprising the amino acid sequence set forth in SEQ ID NO:2.

61. The antibody of clam 57 that specifically binds a SINAT5 polypeptide comprising the amino acid sequence set forth in SEQ ID NO:3.


SEQUENCE LISTING (5 sequences)

SEQ ID NO: 1 - protein, 309 aa, Arabidopsis thaliana
METDSIDSVIDDDEIHQKHQFSSTKSQGGATVVISPATSVYELLECPVCTNSMYPPIHQCHNGHTLCSTCKSRVHNRCPTCRQELGDIRCLALEKVAESLELPCKYYNLGCLGIFPYYSKLKHESQCNFRPYSCPYAGSECAAVGDITFLVAHLRDDHKVDMHTGCTFNHRYVKSNPREVENATWMLTVFQCFGQYFCLHFEAFQLGMAPVYMAFLRFMGDEDDARNYTYSLEVGGSGRKQTWEGTPRSVRDSHRKVRDSHDGLIIQKNMALFFSGGDKKELKLRVTGRIWKEQQNPDSGVCITSMCSS

SEQ ID NO: 2 - protein, 309 aa, Arabidopsis thaliana
METDSIDSVIDDDEIHQKHQFSSTKSQGGATVVISPATSVYELLECPVSTNSMYPPIHQCHNGHTLCSTCKSRVHNRCPTCRQELGDIRCLALEKVAESLELPCKYYNLGCLGIFPYYSKLKHESQCNFRPYSCPYAGSECAAVGDITFLVAHLRDDHKVDMHTGCTFNHRYVKSNPREVENATWMLTVFQCFGQYFCLHFEAFQLGMAPVYMAFLRFMGDEDDARNYTYSLEVGGSGRKQTWEGTPRSVRDSHRKVRDSHDGLIIQKNMALFFSGGDKKELKLRVTGRIWKEQQNPDSGVCITSMCSS

SEQ ID NO: 3 - protein, 309 aa, Arabidopsis thaliana
METDSIDSVIDDDEIHQKHQFSSTKSQGGATVVISPATSVYELLECPVCTNSMYPPIHQCHNGYTLCSTCKSRVHNRCPTCRQELGDIRCLALEKVAESLELPCKYYNLGCLGIFPYYSKLKHESQCNFRPYSCPYAGSECAAVGDITFLVAHLRDDHKVDMHTGCTFNHRYVKSNPREVENATWMLTVFQCFGQYFCLHFEAFQLGMAPVYMAFLRFMGDEDDARNYTYSLEVGGSGRKQTWEGTPRSVRDSHRKVRDSHDGLIIQKNMALFFSGGDKKELKLRVTGRIWKEQQNPDSGVCITSMCSS

SEQ ID NO: 4 - DNA, 1383 bp, Arabidopsis thaliana
ggaaattccagcttcccttttctctctcgttcttgctcttttgatctgcgtgaaaagaaagaatttttattttcccgacagagaaaagtcccaatttttaaaattaggactttttgattttcgaaaattttggtgttaatggaaacagatagtatcgattccgtgatcgatgacgatgagatccatcaaaaacaccaattctcatcaaccaagtctcagggaggagccaccgtggtaatctctccggctacaagcgtttacgagctccttgaatgccctgtctgcaccaattcaatgtacccaccaatccatcagtgtcataatggacatactttatgttcaacctgtaaatcaagagtgcataatcggtgtccaacgtgtagacaagagcttggagatattagatgtttagctcttgagaaagtagctgagtcgcttgagttaccgtgcaagtattacaatcttggatgccttgggattttcccttattacagtaaactaaagcatgagtctcaatgtaactttagaccttatagttgtccatatgctggctcggagtgtgctgctgtaggtgacattactttccttgttgctcatttaagagatgatcataaagtcgatatgcatactggatgtacgtttaatcatcgttatgttaaatctaatccacgcgaagtagagaacgctacttggatgctaacggtgtttcaatgttttggtcaatacttttgtcttcattttgaagcgtttcagctcggtatggctccagtttacatggcgtttctgagattcatgggcgatgaagatgacgcacgaaactatacatacagtttagaagttggaggcagtgggagaaaacagacatgggaagggacaccaagaagtgtcagagatagtcacaggaaagtcagagacagtcatgacggtcttataatccaaaagaacatggcactcttcttttccggtggagacaagaaagaactgaaacttagagtcactggaagaatctggaaagagcaacagaatccagattctggtgtttgcataacctctatgtgtagtagctgaatcaaaatcagccaacccttcaaacctatcttaaggtgttcgttcgatttcttcaattcgattttgtttcgggtttgtgtgttgttttggtccagaatccagatagcttctttacattcaaaagtgtatttagagaagtaaaaagagttgttccatttgccagaaatgtgcagaaggtcacaaaagttgcaataacttccaaagattgaatatttccataaatgtatatattcacatggtaagatctgagcaaagatctaatacagtaacatattcaaatcatatctttatttcattcaaaaaaaaaaaaaaaaa

SEQ ID NO: 5 - protein, 14 aa, Arabidopsis thaliana
TDSIDSVIDDDEIH